(19) United States
(12) Patent Application Publication
(10) Pub. No.: US 2020/0022632 A1
(43) Pub. Date: Jan. 23, 2020

(54) DIGITAL CONTENT PROCESSING AND GENERATION FOR A VIRTUAL ENVIRONMENT

(71) Applicant: Limbix Health, Inc., Palo Alto, CA (US)

(72) Inventors: Benjamin David Lewis, Los Altos, CA (US); Scott Benjamin Satkin, Palo Alto, CA (US)

(21) Appl. No.: 16/513,314

(22) Filed: Jul. 16, 2019

Related U.S. Application Data
(60) Provisional application No. 62/699,414, filed on Jul. 17, 2018.
(60) Provisional application No. 62/699,417, filed on Jul. 17, 2018.

(51) Int. Cl. A61B 5/16; H04N 5/265; H04N 7/01; H04N 9/64; G06T 3/00; H04N 5/232; H04N 5/272; G09B 5/02; G09B 19/00; A61B 5/00; A61B 5/11; A61B 5/0205; A61B 5/0476

(52) U.S. Cl. CPC A61B 5/165; A61B 5/0816; H04N 7/0117; H04N 9/646; G06T 3/0068; H04N 5/23238; H04N 5/272; G09B 5/02; G09B 19/00; A61B 5/7445; A61B 5/6803; A61B 5/1123; A61B 5/02055; A61B 5/0476; H04N 5/265

(57) ABSTRACT

An artificial reality system and method provides immersive digital content to a user via a device with limited capabilities. A digital content processor generates a video stream of a real-world environment in a first video resolution regime. The digital content processor identifies static regions across frames of the video stream. The digital content processor applies one or more of a stitching operation, a blending operation, and a layering operation to replace static regions of the video stream with still image pixels. The digital content processor transmits the modified video stream to a display unit of a virtual reality (VR) device at a second video resolution regime of lower resolution than the first video resolution regime.

### CROSS REFERENCE TO RELATED APPLICATIONS

This application claims the benefit of U.S. Provisional Application Nos. 62/699,414 and 62/699,417, filed Jul. 17, 2018, which are incorporated by reference in their entirety.

### BACKGROUND

The cost of mental health conditions is $1 trillion in the US, with $150 billion spent annually on treatment of mental health conditions. Even though one in five Americans experience symptoms of a mental health condition each year, treatment of mental health conditions is still insufficient in many ways. Typically, there are over six-month long waitlists to receive treatment, treatment options are limited, and therapists are often overworked, which reduces quality of treatment. Use of digital content has been found to be a viable treatment option in relieving therapist burden; however, the use of digital content is often limited due to insufficiencies of devices associated with digital therapeutics and the inability to modify and/or control digital content provided to a patient. In some examples, content quality is limited by insufficiencies in methods and systems to generate high quality content, with respect to output device limitations. Additionally, many current digital therapeutics are not designed to dynamically provide content to a patient via a display device, thus limiting treatment effectiveness.

### SUMMARY

Embodiments relate to a system and method for providing high resolution content to a user for treatment of a mental health condition. Latency and compression issues can arise when attempting to transmit high-resolution video content to users of display devices, for instance, within an artificial reality or virtual reality environment. User perception of such high-resolution content can, however, be appropriate for use in clinical settings for treatment of mental health conditions. In one embodiment, a system includes a virtual reality (VR) platform and a digital content processor for generating and providing digital content to a user.

The digital content processor is configured to modify digital content to generate a video stream for display on a head mounted device associated with the VR platform. The digital content processor receives digital content from one or more sources and generates a video stream in a first video resolution regime. The digital content processor can identify one or more static regions across frames of the generated video stream and substitute one or more pixels associated with a static region with one or more still image pixels. The digital content processor can generate a modified video stream including one or more modified frames comprising still image pixels and video pixels. The digital content processor transmits the modified video stream to the head mounted display unit capable of decoding video content at a second video resolution regime. In one embodiment, the second video resolution regime is associated with a resolution less than that of the first video resolution regime. The system and method allow for transmission of high quality content for display on a device with limited capabilities.

### BRIEF DESCRIPTION OF THE DRAWINGS

FIG. 1 illustrates a system environment for providing VR content to a user, in accordance with one or more embodiments.

FIG. 2 illustrates components of the digital content processor of FIG. 1, in accordance with one or more embodiments.

FIG. 3 depicts a schematic of an example digital content capture system, in accordance with one or more embodiments.

FIG. 4A illustrates content that may be captured by the digital content capture system, in accordance with one or more embodiments.

FIG. 4B illustrates an example of modified content, in accordance with one or more embodiments.

FIG. 4C illustrates an example frame of modified video content, in accordance with one or more embodiments.

FIG. 5A illustrates a first example environment for treatment of a mental health condition, in accordance with one or more embodiments.

FIG. 5B illustrates a second example environment for treatment of a mental health condition, in accordance with one or more embodiments.

FIG. 5C illustrates a third example environment for treatment of a mental health condition, in accordance with one or more embodiments.

FIG. 5D an example layer for treating a mental health condition, in accordance with one or more embodiments.

FIG. 5E illustrates the example environment of FIG. 5A with the layer of FIG. 5D, in accordance with one or more embodiments.

FIG. 6 illustrates an example flow of digital content segmentation, in accordance with one or more embodiments.

FIG. 7 is a flowchart illustrating a method of generating video content for presentation to a user, in accordance with one or more embodiments.

FIG. 8 is a flowchart illustrating a method of dynamically modifying digital content for presentation to a user, in accordance with one or more embodiments.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

### Overview

Millions of people struggle with mental health conditions, for example, depression, PTSD, anxiety, etc. Mental health conditions affect individuals of all ages and genders, although some illnesses disproportionately affect certain age groups. For example, teens are disproportionately affected by depression. It may be difficult to engage patients, especially teens, in certain aspects of treatment for mental health conditions. Additionally, treatment for mental health conditions can be expensive and time consuming because of the limited resources. Digital content that provides a more immersive environment could be valuable for treating mental health patients, but there are constraints on the content that can be provided given technical limitations associated with video display devices (e.g., a virtual reality headset). Additionally, digital content used to treat patients may be difficult to modify, especially in real-time, such that the digital content can be tailored or customized to a particular patient or mental health condition.

Generally, patients are treated for mental health conditions using therapy administered by a clinician. Examples of therapies that may be administered by a clinician include cognitive behavioral therapy, talk therapy, interpersonal therapy, behavioral activation therapy, exposure therapy, psychoanalysis, humanistic therapy, and many other types of therapies. In some example digital therapeutic systems that may be integrated with one of these types of therapies, the therapist often cannot adjust content according to a patient's response to better tailor the content to the particular patient over the course of treatment such that the patient receives treatment personalized to him or her. Additionally, existing mobile applications used in treatment of mental health conditions are designed to replace therapists as opposed to helping therapists treat patients more efficiently and effectively. Accordingly, a system that allows the patient to engage with the content in a more immersive or experiential manner while the patient is interacting with the content may be beneficial in treating mental health conditions. In one embodiment, a virtual reality (VR) platform can be used to provide one or more types of therapy to a patient in addition to or instead of a therapist, thus increasing patient engagement and reducing therapist burden. In one embodiment, a system for treating a patient includes a VR platform, a client device, a clinician device, a digital content capture system, a digital content processor, and a state detection system.

The VR platform may have limited capabilities in providing content to a user via a head mounted display such as limited processing speed, limited storage space, limited video size, etc. For example, typically 8K video requires 1-2 GB/minute of storage, and most VR platforms may not have this capacity. Thus, the digital content processor can modify video data to present a video stream to a patient using the VR platform such that the patient can experience high quality content even with limited capabilities of the VR platform. In one embodiment, a digital content processor modifies a video stream in a first resolution (e.g., 8K resolution) such that it can be displayed on a device with a lower resolution (e.g., 4K resolution).

Digital content can be used to treat mental health conditions, engage a user's attention, and/or for any other suitable purpose. For example, for users who experience vertigo or have a fear of heights, generated content for a VR environment can “place the users” at the top of a tall building or other location where the user can face the fear of heights without actual danger to the user. In another example, for users with post-traumatic stress disorder (PTSD) or even users with situational anxiety (e.g., a fear of a particular situation, such as a social situation or a crowded area), generated content for the VR environment can “place the users” in situations that promote treatment of the anxiety condition or PTSD (e.g., through exposure therapy). In this embodiment, the system allows for display of high resolution video on mobile devices with limited computation and storage. In some embodiments, the system generates video content for an immersive, artificial reality or virtual environment (e.g., virtual reality (VR), augmented reality (AR), mixed reality (MR), cross reality (XR), etc.), allowing a user to experience high quality content pertaining to different situations or environments for therapeutic reasons. Generation of high quality content without succumbing to computation power issues associated with latency, compression, resolution, storage, or other issues is also relevant to other applications.

In some embodiments, it may be beneficial to provide dynamic content to a patient, such that the digital content can be adjusted specifically to the user. Thus, the digital content processor can modify digital content based on information received from the state detection system and/or the clinician device. The state detection system can include one or more sensors and/or subsystems for detecting information related to one or more states of a patient. The digital content processor can modify the digital content to increase or decrease anxiety levels of a user to enhance patient treatment.

Dynamic delivery of content, in response to user behaviors and/or other factors can be especially effective in improving treatment outcomes for mental health conditions since the content delivered can be controlled and adjusted over the course of treatment based on the response of the patient to the content. The method(s) and/or system(s) described herein can be used to provide digital content to users, automatically detect when a feature of the digital content should be modified (e.g., based on a user behavior, based on decisions made by an outside administrator, etc.), modify the feature, and seamlessly deliver modified digital content with the modified feature to the user, without perceptible artifacts due to, for instance, stitching processes, blending processes, latency issues, and compression issues associated with streaming digital content. The method(s) and/or system(s) described herein can be further adapted to be used in a clinical setting in relation to providing solutions to problems related to wireless streaming limitations, portability, usability, adaptation to use by various users, and other problems.

### 1. System Environment

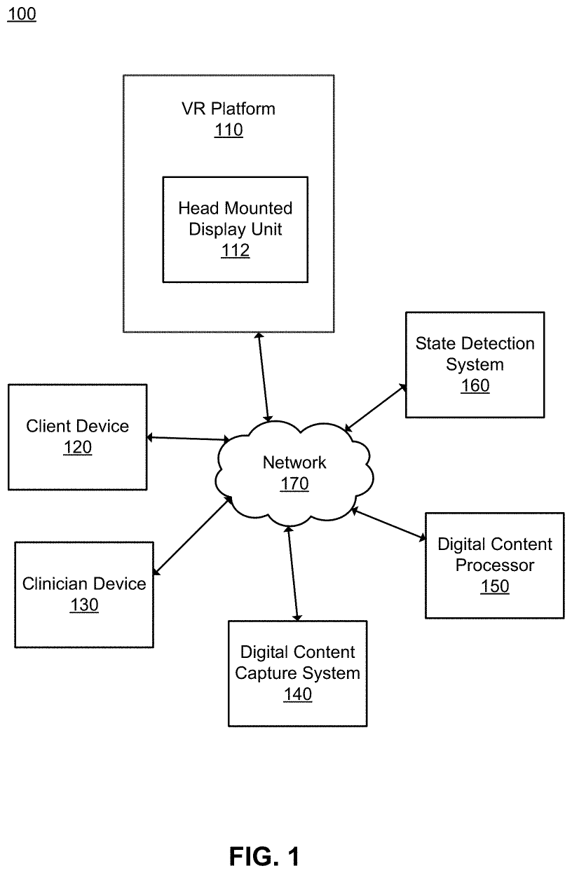

FIG. 1 depicts a system environment 100 for providing treatment for a mental health condition to a patient, in accordance with an embodiment. The system 100 shown in FIG. 1 includes a VR platform 110, a client device 120, a clinician device 130, a digital content capture system 140, a digital content processor 150, and a state detection system 160 connected via a network 170. In alternative configurations, different and/or additional components may be included in the system environment 100. Additionally, the components may have fewer or greater features than described herein. For example, the components 140, 150, 150 can be included within a single entity or a single server, or across multiple servers. As another example, the state detection system may not be present in embodiments that are not focused on determining the state of a user.

The VR platform 110 can include one or more devices capable of receiving user input and transmitting and/or receiving digital content. The VR platform 110 can provide different types of digital content to a user including video content, audio content, image content, and any other suitable types of content for treating a user for a mental health condition. Additionally, the VR platform 110 may be configured to provide haptic content or feedback to a user. In some embodiments, the VR platform 110 receives digital content from the digital content capture system 140, the digital content processor 150, or some combination thereof for presentation to the user. The term “VR platform” is used throughout, but this can include other types of immersive, artificial reality platforms, such as AR, MR, XR, etc.

In one embodiment, the VR platform 110 includes a head mounted display (HMD) unit 112 configured to display digital content. The HMD can be goggles, glasses or other device that is mounted on or strapped to the user's head or worn by the user, including devices mounted on or in a user's face, eyes, hair, etc. The HMD can be a custom designed device to work with the VR platform. In some embodiments, HMDs available by various venders can be used, such as an OCULUS device (RIFT, QUEST, GO, etc.), NINTENDO LABO, HTC VIVE, SAMSUNG GEAR VR, GOOGLE DAYDREAM, MICROSOFT HOLOLENS, etc. The HMD unit 112 can operate in a lower resolution regime than the regime in which video content is generated (e.g., by the digital content processor 150). Additionally, the VR platform 110, with the HMD unit 112, can be associated with a frame rate (e.g., from 6 frames per second to 200 frames per second), aspect ratio or directionality (e.g., unidirectionality), format (e.g., interlaced, progressive, digital, analog, etc.), color model, depth, and/or other aspects. The HMD unit 112 can be configured to display monoscopic video, stereoscopic video, panoramic video, flat video, and/or any other suitable category of video content. In a specific example, the VR platform 110, with the HMD unit 112, is configured to decode and transmit 3K or 4K video content (in comparison to 8K video content generated in by the digital content processor 150 and/or the digital content capture system 140, described in greater detail below). The VR platform 110, with the HMD unit 112, also has a 90 Hz framerate, audio output capability, memory, storage, and a power unit. In some examples, the VR platform 110 can include a storage component configured to store video content, such that content can be delivered in a clinical environment less amenable to streaming from an online source or other remote source. Alternatively, the VR platform 110 can display high resolution video with other hardware that can decode the high-resolution video, in a manner that decreases bandwidth and storage requirements for the hardware displaying the video.

The VR platform 110 can additionally or alternatively include one or more of: controllers configured to modulate aspects of digital content delivered through the HMD unit 112, power management devices (e.g., charging docks), device cleaning apparatus associated with clinical use, enclosures that retain positions of a device (e.g., HMD unit 112, control device/tablet, audio output device, etc.), handles to increase portability of the VR platform 110 in relation to use within a clinical setting or other setting, medical grade materials that promote efficient sanitization between use, fasteners that fasten wearable components to users in an easy manner, and/or any other suitable devices. In one embodiment, the VR platform 110 is provided as a kit that includes one or more components useful for a therapist or coach to provide a VR experience for a user. The kit can include a control device, such as a tablet, an HMD unit 112 for the user to wear, headphones, chargers and cables (e.g., magnetic charging cables to prevent micro USB breakage and allow the kit to be set up more quickly, etc.). Elements of the kit can be held in a portable light-weight enclosure, such as a briefcase or bag. The HMD unit 112 can have a custom designed, adjustable fully medical grade head strap to fit the user properly (e.g., with a ratchet handle) to make it easier for the administrator to put the device on the user and sanitize afterwards.

The VR platform 110 and the HMD unit 112 can be configured to provide a variety of environments to a user for treating mental health conditions. For example, the VR platform 110 may provide an environment configured to treat anxiety, depression, PTSD, substance abuse, attention deficit disorder, eating disorders, bipolar disorder, etc. Example treatment environments for different mental health conditions include: a vehicle operation environment for treating vehicle related anxiety (e.g., operating a vehicle in various traffic situations, operating a vehicle in various weather conditions, operating a vehicles with varying levels of operational complexity, etc.); a social situation environment for treating social anxiety (e.g., interacting with others in a party environment, interacting with varying numbers of strangers, etc.); a fear-associated environment (e.g., an environment associated with acrophobia, an environment associated with agoraphobia, an environment associated with arachnophobia, etc.); and any other suitable environment associated with user stressors. In other variations, the VR platform 110 environments associated with relaxation or distractions from present stressful, pain-inducing, or anxiety-causing situations (e.g., such as visually-pleasing environments tailored to relax a user who is undergoing a clinical procedure). In other variations, the environments can include environments associated with training of skills (e.g., meditation-related skills) for improving mental health states.

In other embodiments, the VR platform 110 can provide environments useful for diagnostic applications (e.g., environments where the user's focus can be assessed to diagnose autism spectrum disorder conditions). In still other variations, the VR platform 110 can provide content presenting environments associated with mindfulness exercises. Additionally, environments may be selected based on diagnostic and/or therapeutic methods described in manuals of mental health conditions (e.g., The Diagnostic and Statistical manual of Mental Disorders, Chinese Classification and Diagnostic Criteria of Mental Disorders, Psychodynamic Diagnostic Manual, etc.). The VR platform 110 can provide any other suitable environment(s) for treating one or more health conditions.

The VR platform 110 communicates with the client device 120 and/or the clinician device via the network 170. In some embodiments, the devices 120 and 130 are part of the VR platform 110 and may include software specialized to the VR platform 110. In other embodiments the devices 120 and 130 are separate from the VR platform 110. The client device 120 may be monitored/used by a patient, while the clinician device 130 may be monitored by a therapist, coach, a medical professional, or another individual. The client device 120 and the clinician device 130 may be a mobile device, a laptop, a computer, a tablet, or some other computing device. In some cases, the client device 120 and/or the clinician device 130 can download a mobile application that may be synched with the VR platform 110. The mobile application can provide a user interface in which the user and/or the clinician can view and analyze results from user interaction with the VR platform 110. Additionally, the mobile application can be available for use on other devices (e.g., of family and friends, physicians, etc.).

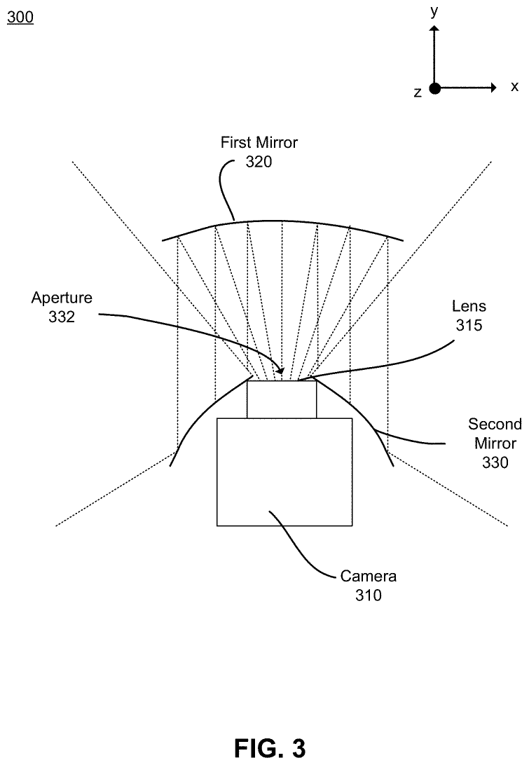

The digital content capture system 140 is configured to capture video and/or images of a real-world environment. The digital content capture system 140 may include one or more devices configured to capture images and/or video. Examples of devices include, but are not limited to, stereoscopic cameras, panoramic cameras, digital cameras, camera modules, video cameras, etc. The digital content capture system 140 can include one or more devices mounted to an object that is static relative to the environment of interest. Alternatively, the content capture system 140 can be mounted to an object moving within the environment of interest. In one embodiment, the digital content capture system 140 includes an omnidirectional camera system, described below in relation to FIG. 3. The digital content capture system 140 can additionally or alternatively include any other suitable sensors for generating digital audio content, haptic outputs, and/or outputs associated with any other suitable stimulus.

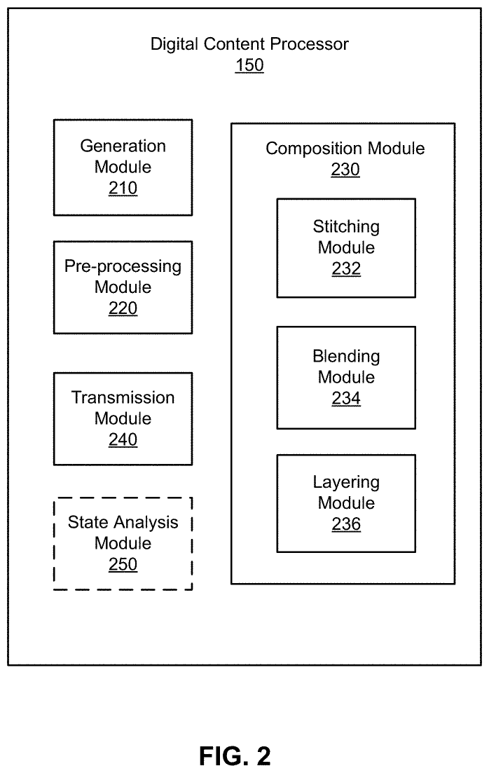

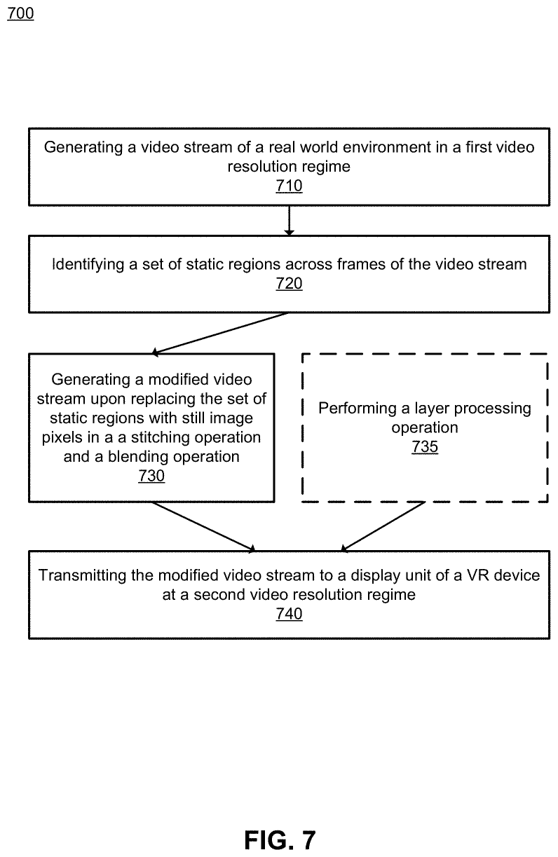

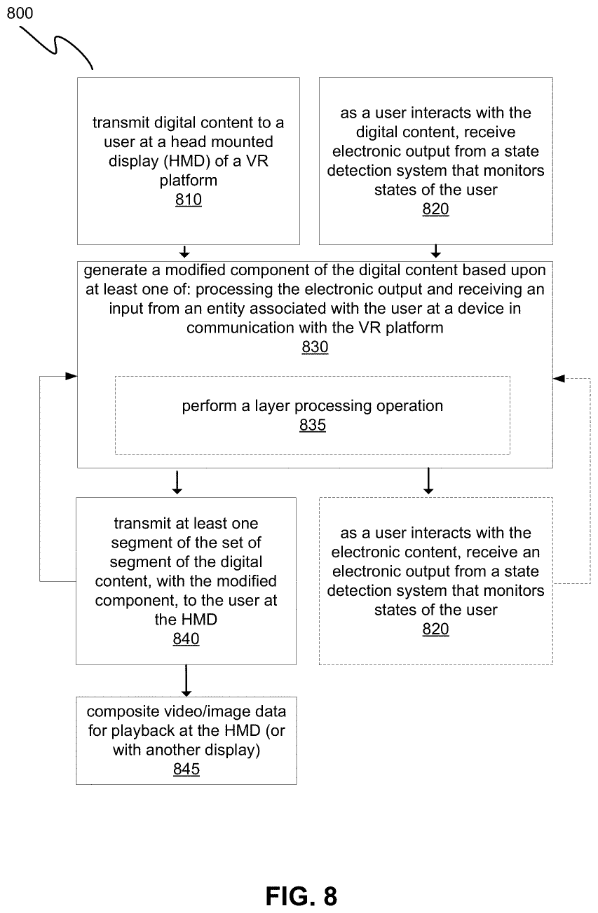

The digital content processor 150 is configured to process digital content (e.g., video data) and provide modified digital content (e.g., video streams) to VR platform 110 for presentation to a user. The digital content processor 150 can receive digital content from the digital content capture system 140 or some other system that captures, generates, and/or stores digital content. The digital content processor 150 can include computing subsystems implemented in hardware modules and/or software modules associated with one or more of: personal computing devices, remote servers, portable computing devices, cloud-based computing systems, and/or any other suitable computing systems. Such computing subsystems can cooperate and execute or generate computer program products comprising non-transitory computer-readable storage mediums containing computer code for executing embodiments, variations, and examples of methods described below in relation to FIG. 7 and FIG. 8. Components of the digital content processor 150 are shown in FIG. 2 and described in greater detail below.

The state detection system 160 is configured to monitor one or more states of a user. The state detection system 160 includes one or more subsystems and/or one or more sensors for detecting aspects of user cognitive state and/or behavior as users interact with the VR platform 110. The state detection system 160 can include audio subsystems and/or sensors (e.g., directional microphones, omnidirectional microphones, etc.), optical subsystems and/or sensors (e.g., camera systems, eye-tracking systems) to process captured optically-derived information (associated any portion of an electromagnetic spectrum), and motion subsystems and/or sensors (e.g., inertial measurement units, accelerometers, gyroscopes, etc.). The state detection system 160 can additionally or alternatively include biometric monitoring sensors including one or more of: skin conductance/galvanic skin response (GSR) sensors, sensors for detecting cardiovascular parameters (e.g., radar-based sensors, photoplethysmography sensors, electrocardiogram sensors, sphygmomanometers, etc.), sensors for detecting respiratory parameters (e.g., plethysmography sensors, audio sensors, etc.), brain activity sensors (e.g., electroencephalography sensors, near-infrared spectroscopy sensors, etc.), body temperature sensors, and/or any other suitable biometric sensors. In some embodiments, the state detection system 160 is configured to process information associated with a user's interactions with the VR platform 110 and/or entities associated with the user (e.g., therapist). The state detection system 160 can generate electrical outputs based on the information and provide the outputs to the digital content processor 150. In other embodiments, the state detection system 160 provides the information to the digital content processor 150, and the digital content processor 150 analyzes the captured data. The digital content processor 150 processes the electrical outputs of state detection system 160 to extract features useful for guiding content modification in near-real time.

In one embodiment, the state detection system 160 can be coupled to, integrated with, or otherwise associated with the VR platform 110. The state detection system 160 can additionally or alternatively be coupled to, integrated with, or otherwise associated with components of the digital content processor 150 in proximity to the user during interactions between the user and provided digital content. Sensors of the state detection system 160 can alternatively interface with the user in any other suitable manner and communicate with the components of the system 100 in any other suitable manner.

The network 170 is configured support communication between components of the system 100. The network 170 can include any combination of local area and/or wide area networks, using both wired and/or wireless communication systems. In one embodiment, the network 170 uses standard communications technologies and/or protocols. For example, the network 170 includes communication links using technologies such as Ethernet, 802.11, worldwide interoperability for microwave access (WiMAX), 3G, 4G, code division multiple access (CDMA), digital subscriber line (DSL), etc. Examples of networking protocols used for communicating via the network 170 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over the network 170 may be represented using any suitable format, such as hypertext markup language (HTML) or extensible markup language (XML). In some embodiments, all or some of the communication links of the network 170 may be encrypted using any suitable technique or techniques.

### 2.0 Digital Content Processor

The digital content processor 150 is configured to generate and modify digital content (e.g., a video stream) for presentation of the digital content on a user device. In one embodiment, the digital content processor 150 can modify a video stream such that the video stream can be displayed on a device with limited computation power and storage (e.g., VR platform 110). In a specific example, the digital content processor 150 can generate and modify 8K video such that it can be displayed on an HMD unit 112 that can only decode 4K video. In some embodiments, the digital content processor 150 performs real-time rendering of video content in a manner that can be decoded by the display unit of the VR platform 110. The digital content processor 150 can divide the video stream into one or more segments and modify the segments responsive to information from the state detection system 160 and/or the clinician device 130. Alternatively, the digital content processor 150 may modify an entire video stream before the digital content processor 150 provides the video stream to the VR platform 110.

The digital content processor 150 includes a generation module 210, a pre-processing module 220, a composition module 230, a transmission module 240, and a state analysis module 250. The modules are configured to process digital content for presentation to a user via the VR platform 110. The digital content processor 150 can generate different types and formats of video content and the video content can include a range of environments. In one embodiment, the digital content processor 150 generates and transmits video content that presents a human stress-associated environment, which can be used to treat mental health conditions, with or without involvement of a therapist. For example, for a user with social anxiety, the user can be provided with content that immerses the user in a particular situation that induces anxiety, such as an environment in which the user is surrounded by a social situation that makes the user uncomfortable. In some embodiments, a video stream includes in one or more segments, and the segments are divided based on a treatment plan. For example, the video stream can include segments for different treatment purposes (e.g., an introductory segment, a meditation segment, a treatment segment, a stress relieving segment). In some embodiments, the digital content processor 150 modifies the order of the segments as the user experiences the video stream using the VR platform 110.

The modules of the digital content processor 150 are configured to seamlessly transmit different environments to a patient such that the patient can be treated for one or more mental health conditions. In some embodiments, the digital content processor 150 is configured to generate video that follows a treatment plan specific to the user, described in greater detail below. The digital content processor 150 may modify and/or transmit video content responsive to input from the client device 120, the clinician device 130, and/or the state detection system 160.

### 2.1 Generation Module

The generation module 210 is configured to generate an initial video stream using digital content received from the digital content capture system 140, a storage system, additional content capture systems, or some combination thereof. The digital content may be in a variety of formats or categories. In one embodiment, the digital content capture system 140 provides video and/or image data from a real-world environment, as described above in relation to FIG. 1. In other embodiments, the generation module 210 receives video data from another system to generate a rendered or animated video stream. The generation module 210 can generate stereoscopic video, panoramic video, monoscopic video, volumetric video, narrow field of view video, flat video, and/or any other suitable category of video content. Properties of the generated video stream can include resolution (e.g., 8K, 4K, 2K, WUXGAm 1080p, 720p, etc.), frame rate (e.g., from 6 frames per second to 200 frames per second), aspect ratio or directionality (e.g., unidirectionality), format (e.g., interlaced, progressive, digital, analog, etc.), color model, depth, audio properties (e.g., monophonic, stereophonic, ambisonic, etc), and/or other aspects.

In one embodiment, the generation module 210 generates a video stream in a first video resolution regime (e.g., 8K resolution) which is subsequently modified by the composition module 230 to a second video resolution regime (e.g., 4K resolution), described in greater detail below. The first video resolution regime can function as baseline high-resolution content. The baseline content can be used to generate an immersive, realistic environment. Additionally, the generation module 210 can generate other forms of digital content that can be provided within a VR platform 110. For instance, the generation module 210 can additionally or alternatively generate audio content, text content, haptic feedback content, and/or any other suitable content (e.g., content complementary to the video content). The generation module 210 can also be configured to generate and/or design non-digital stimuli (e.g., olfactory stimuli, taste sensation stimuli, etc.) that may be provided to the user by the VR platform 110.

### 2.2 Pre-Processing Module

The generated video stream can include a plurality of frames, where each frame includes static regions that remain essentially unchanged from frame to frame and motion associated regions (referred to herein as “dynamic regions”) that change from frame to frame. In some embodiments, the frames may be grouped into segments, where each segment includes a plurality of consecutive frames. The pre-processing module 220 is configured to identify one or more static regions in one or more frames of the generated video stream. The pre-processing module 220 can additionally or alternatively identify one or more dynamic regions in one or more frames of the video stream. In one embodiment, the pre-processing module 220 receives a generated video stream from the video generation module 210 and identifies static regions to define regions of interest (e.g., pixel boundaries within frames of video data) within video data that can be replaced with image data. The pre-processing module 220 may label the static regions (e.g., as background content, as static content). The pre-processing module 220 may identify static regions in each frame of the video stream, in each frame during one or more segments of the video stream, in each frame at the beginning and end of the video stream, or in any suitable frames. The pre-processing module 220 can also identify temporal parameters associated with the static regions in each frame to identify these regions, such that motion can be characterized both spatially and temporally (e.g., within and/or across video frames).

In some embodiments, the pre-processing module 220 can implement one or more computer vision techniques for identifying changes in position of features captured in frames of the generated video stream. In variations, the pre-processing module 220 can employ one or more of: optical flow techniques, background subtraction techniques (e.g., Gaussian Mixture Model-based foreground and background segmentation techniques, Bayesian-based foreground and background segmentation techniques, etc.), frame difference-based computer vision techniques, and temporal difference-based computer vision techniques. The pre-processing module 220 can function without manual annotation or adjustment to identified regions of interest, or can alternatively be implemented with manual annotation/adjustment. In a specific example, the pre-processing module 220 can implement an optical flow process (e.g., Lucas Kanade technique, Horn-Schunck technique) and a process that computes bounding boxes for the dynamic regions of the video stream. The pre-processing module 220 can store descriptions of the dynamic regions in a video description file. The pre-processing module 220 can extract video portions as needed based upon the video description file. In relation to storage, the pre-processing module 220 can write to a separate file each video portion or package and store together all video portions as a texture atlas of video frames by spatially concatenating all regions of each extracted video frame.

### 2.3 Composition Module

The composition module 230 is configured to create a video stream based on the identified static regions. The composition module 230 can receive the generated video stream from the pre-processing module 220 and replace one or more static regions identified by the pre-processing module 220 with still image pixels. The composition module 230 may replace the static regions using a single substitute image or from multiple substitute images. The substitute image may be received from the digital content capture system 140, another content capture system, from a storage module, or from any other suitable component. The substitute image may have a smaller resolution than the video (e.g., 4K resolution) such that replacement of the static pixels with the still images reduces the resolution of the video stream. The composition module 230 includes a stitching module 232, a blending module 234, and a layering module 236 to facilitate the process of merging dynamic regions in the video stream with still image pixels, described below. The stitching module 232, blending module 234, and layering module 236 may operate collectively, in parallel, subsequent to one another, or some functions may be distributed differently than described herein.

In one embodiment, the composition module 230 merges the dynamic regions of one or more frames of the video stream by overlaying the dynamic regions on a still image. As such, the composition module 230 can process dynamic regions into one or more 2D textures (e.g., equirectangular, cube map projections). Regions of a video stream may be represented by a rectangle in 2D image coordinates. In one embodiment, the composition module 230 decodes frames associated with each dynamic region with a separate media player and processes each dynamic region into its own 2D texture. Alternatively, the composition module 230 can decode all dynamic regions with a single media player and process them into a single 2D texture. The composition module 230 can extract the coordinates of each dynamic region (or conversely, coordinates of static regions) from the video description file.

### 2.3.1 Stitching Module

The stitching module 230 transforms a still image to fit the static regions of a video stream. In one embodiment, the stitching module 230 implements a homography matrix operation to transform features of a still image in order to fit the static regions of the video stream with minimal perceived distortion. The stitching module 230 can additionally or alternatively apply one or more of: an image alignment process that relates pixel coordinates of the substitute image(s) to pixel coordinates of static regions of video content and estimates correct alignments across collections of images/frames of the video stream, a feature alignment process that determines correspondences in features across images images/frames of the video stream (e.g., using key point detection), registration processes, and any other suitable image stitching process.

In one variation, the stitching module 230 converts pixel coordinates corresponding to the still image and/or video stream to a 2D texture. For example, a still image includes a plurality of pixels, and each pixel corresponds to a ray in a 3D space. The stitching module 230 can convert each ray having 3D cartesian coordinates to 2D coordinates (e.g., polar coordinates in [Om] space). Generally, the 2D coordinates have a linear correspondence to pixels in the equirectangular texture. As such, the coordinates of the equirectangular texture may correspond to a 2D texture coordinate of the generated video content. Based on the coordinates, the stitching module 230 can determine whether the ray intersects a video region to determine if a pixel of a substitute still image corresponding to the ray should be stitched into a video frame to replace a static portion of the video frame. In this assessment, the stitching module 230 can convert the ray's coordinates (e.g., polar coordinates) to a video frame coordinate space (e.g., a [0,1] space), and can assess whether or not the coordinates of the ray fall within the video frame coordinate space. If the coordinates of the ray fall within the video frame coordinate space, the stitching module 230 can index the pixel corresponding to the ray into the corresponding video content frame at the computed coordinates in frame coordinate space and can process with the blending operation described below.

### 2.3.2 Blending Module

The blending module 234 blends still image pixels replacing the static regions and other portions of the video frame. In one variation, the blending module 234 blends the video frame with pixels of the substitute still image(s) at boundaries between dynamic regions and still image portions. Blending at the boundaries may be subject to a condition associated with a fraction of the size of the video frame. The fraction condition may prevent blended regions from encompassing beyond a threshold fraction of the video frame. In one variation, the blending module 234 can compute a frame weight that factors in the threshold fraction, and produce an output pixel corresponding to a blending region, based on the frame weight.

In one embodiment, blending module 234 blends images and video only where there is motion, such that the system maximizes the amount of content that is image content as opposed to video content. The blending module 234 enables an arbitrarily high resolution video to be displayed on a mobile or other device with limited computation power and storage. In some embodiments, the blending module 234 determines a color value of the pixel of the substitute image replacing the static portion of the video frame, and smooths boundaries between substitute image pixels and dynamic regions based the color value. The blending module 234 can determine the color value to minimize or eliminate perceived color distortion associated with blended images, in relation to environmental lighting associated with the video content. Determining the color value can include correcting color for each color channel in RGB space, CMYK space, PMS space, HEX space, and/or any other suitable space. In variations, determining the color value can include implementing a color correction operation for each pixel including one or more of: a color grading operation, an irradiance correction operation, a Gamma correction operation (e.g., for luminance components), a linear correction operation (e.g., for chrominance components), a gradient domain operation, a geometric-based color transfer operation, a statistics-based color transfer operation, and any other suitable color matching operation. In some embodiments, the blending module 234 takes advantage of knowledge of the scene geometry (e.g., 3D models, depth maps, surface normal) and lighting (e.g., 3D models of lights, spherical harmonics, environment maps) to make synthetically inserted objects appear as if they are truly in the environment. For example, the blending process can simulate how the environmental lighting influences the objects appearance and how shadows are cast on an inserted object.

### 2.3.3 Layering Module

The layering module 236 can perform one or more layer processing operations on the digital content (e.g., involving decomposition and processing of different regions of video/imagery). The layering module 236 functions to support inclusion of additional layers of still or dynamic content in the modified video stream. The layers of the content can be associated with different components or features (e.g., anxiety-inducing elements having different levels of severity). In variations, described in more detail below, layered video content can include a first video layer capturing a first entity (e.g., coach within a VR environment) or environmental aspect (e.g., stress-inducing aspect) associated with a therapy regimen, a second video layer capturing a second entity or environmental aspect, and/or any other suitable layers aggregated in some manner. The composition module 230 can composite layers of video and/or image data prior to playback at the VR platform 110 or other display, for presentation of modified content to a user including one or more layers.

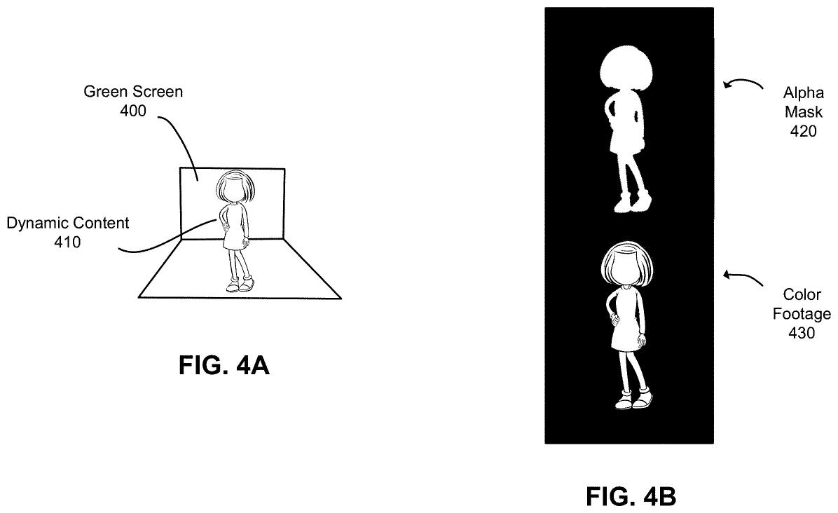

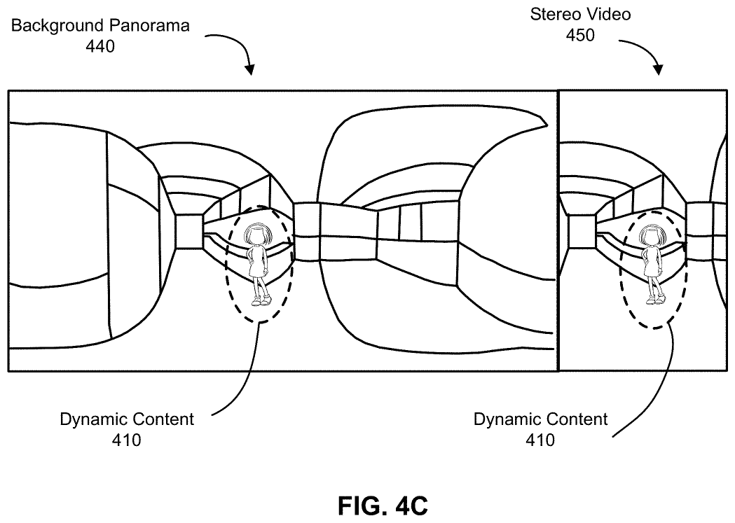

In one embodiment, the layering module 236 can perform modified stitching and blending operations in addition to or instead of the operations discussed in relation to the stitching module 232 and the blending module 234. In one variation, the layering module 236 processes video content of an additional layer to compute a foreground mask (e.g., an alpha mask determined based on a chromakey background subtraction operation), as shown in FIG. 4B and described below. The layering module 236 can spatially concatenate the foreground mask with baseline video content or other intermediate stages of processed video content derived from the video stream. The layering module 236 can compute a weight of each video frame by combining (e.g., multiplying) a weight parameter associated with the frame with an alpha value of a corresponding pixel in the foreground mask. The weight can be used to guide blending operations, determine computational requirements associated with decoding and delivery of processed video content, and/or for any other suitable purpose.

Additionally, the layering module 236 can determine color (e.g., average luminance) characteristics in a layer(s) of the digital content, and can match color characteristics in layers associated with the modified content based on the characteristics of the layer(s). Similar to the blending module 234, the layering module 236 determines the color characteristics functions to minimize or eliminate perceived color distortion associated with different layers in relation to environmental lighting associated with the digital content. The layering module 236 can implement color correction and/or blending operations by correcting color for each color channel in RGB space, CMYK space, PMS space, HEX space, and/or any other suitable space. Color correction operations can include a color grading operation, an irradiance correction operation, a Gamma correction operation (e.g., for luminance components), a linear correction operation (e.g., for chrominance components), a gradient domain operation, a geometric-based color transfer operation, a statistics-based color transfer operation, or any other suitable color matching operation.

In one variation, in addition to a color correction operation, the layering module 236 applies a color grading operation to a foreground layer and determine a color value of each foreground pixel subsequent to color grading operation performed by the blending module 234, thereby simulating illumination of the foreground layer by the background. The layering module 236 can determine an average irradiance that characterizes average illumination across a frame of video content, and process the foreground layer with the average irradiance and a factor that defines a ratio of foreground-to-background blending occurs during the color grading operation.

In one embodiment, the layering module 236 processes layers configured for treatment of specific mental health conditions. For example, the content can include layers associated with different types of social situation (e.g., party situation, one-on-one situation, public speaking situation, etc.), layers associated with number of individuals (e.g., a few individuals, a group of individuals, a crowd of individuals, etc.), types of individuals (e.g., relatives, acquaintances, strangers, etc.), and/or any other suitable layers. In a more specific example, the user might first be provided with a layer in which the user is in a room with various people that do not interact with the user, but then the user might be provided a layer in which a person attempts to interact with the user, which may be a higher stress situation for the user. In other examples, layers can include layers associated with one or more of: acrophobia, agoraphobia, arachnophobia, cynophobia, phonophobia, zoophobia, and/or any other suitable fear. In other variations, the layers can be associated with environments for providing relaxation or distractions from present stressful, pain-inducing, or anxiety-causing situations. In one example, one or more layers of video content can capture aspects of visually-pleasing environments tailored to relax a user who is undergoing a clinical procedure, wherein visually pleasing environments can have layers associated with varying levels of pleasant scenery components.

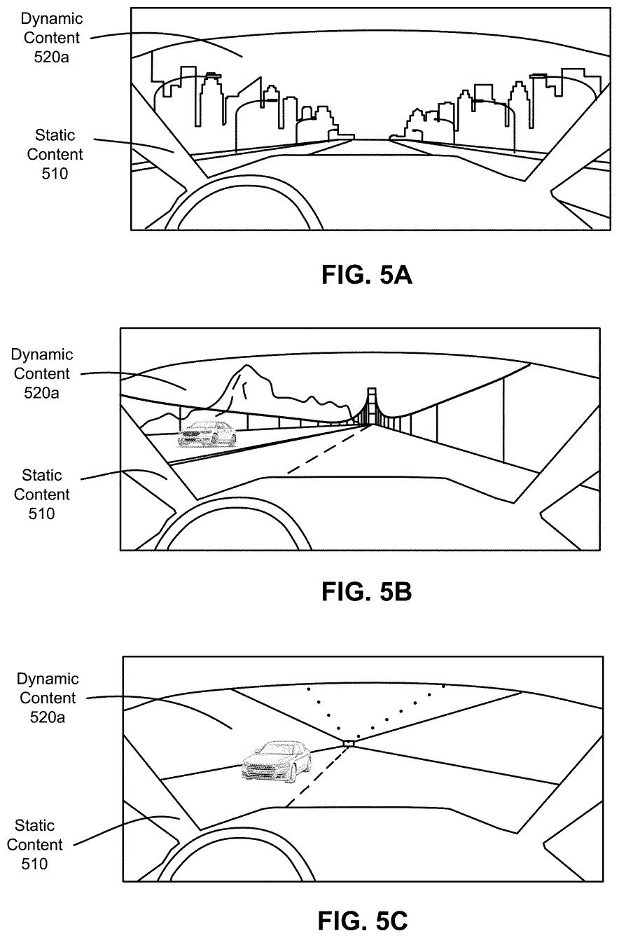

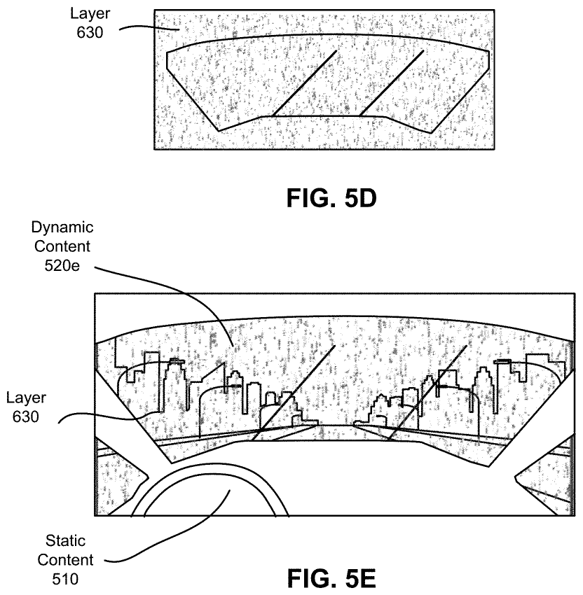

Variations of layer processing can also include one or more visual effects layers for scene customization. Visual effects layers can be used to further adjust how much anxiety a user is exposed to, in relation to an environment presented to a user in modulated content. In examples, visual effects layers can add one or more of: weather effects (e.g., rain, lightning, wind, fog, etc.), explosions, projectiles, fire, smoke, falling objects, obstacles, or any other suitable visual effect to a scene. The layering module 236 can apply one or more layer processing operations based on information from the state detection system 160 and/or the clinician device 130. Examples of layering operations are described below in relation to FIG. 5A-5E.

### 2.4 Transmission Module

The transmission module 240 transmits the modified video stream to a display unit (e.g., HMD unit 112) of the VR platform 110 capable of decoding video content at a second video resolution regime. The second video resolution regime can be of lower resolution (e.g., 4K resolution) than the first video resolution regime, or can alternatively be of the same or greater resolution than the first video resolution regime. The transmission module 240 transmits a modified video stream to reduce computation and storage requirements associated with decoding the modified video stream. As such, transmission module 240 delivers modified video content through a device having decoding and/or transmission limitations, such that the user perceives the content as being higher resolution content than can typically be delivered through such systems. The transmission module 240 can transmit the modified video stream by systems coupled to the VR platform 110 or display device through one or more hardware interfaces. Furthermore, the digital content processor 150 can process and composite video/image data during presentation or playback of content to the user at the VR platform 110 (e.g., the digital content processor 150 can operate in real-time), and the transmission module 240 may continuously transmit video content to the VR platform 110. Additionally, or alternatively, the digital content processor 150 can store content on the VR platform 110 or other device associated with a display that may be accessed in real time (or near real time) in relation to a user session where content is provided to the user.

In one embodiment, the transmission module 240 transmits multiple segments of digital content having modified components (e.g., variations in decomposited and composited layers). The transmission module 240 can implement a latency reduction operation that employs a set of video decoders with frame buffers to improve transitions between segments of digital content provided to the user. In one variation, the transmission module 240 can include loading a subsequent segment of digital content (e.g., including the modified component), with one of the set of video decoders and frame buffers while a currently playing segment is being delivered through the HMD unit 112. Then, once the subsequent segment is loaded to a degree that satisfies a threshold condition, the transmission module 240 can switch from the frame buffer for the currently playing segment to the frame buffer for the subsequent segment.

At a frame level, the transmission module 240 can also include implementation of a frame analysis operation that analyzes similarity between one or more frames prior to and after a transition point between segments of provided content in order to determine if transitioning between segments of content at the transition point will be satisfactorily seamless. The frame analysis operation can include performing a pairwise distance or similarity analysis that outputs a similarity metric between frames associated with the transition point, where the pairwise distance or similarity analysis analyzes characteristics of the frames. The characteristics can include color characteristics of single pixels, color characteristics of multiple pixels, color characteristics of a frame, motion characteristics across frames, coordinates of a frame element across frames, and/or any other suitable characteristics. In one variation, the pairwise distance/similarity analysis can determine an RGB color difference between a pre-transition frame and a post-transition frame (e.g., by performing pairwise comparisons across pixels of the frame not associated with modulated content) to determine if the transition will be satisfactorily smooth from a color perspective. The pairwise distance/similarity analysis can also determine differences in scene motion across frames using optical flow methods to determine if the transition will be satisfactorily smooth from a motion perspective. In some cases, the transmission module 240 analyzes rectangular coordinates of a point or feature across a pre-transition frame and a post-transition frame to determine if a trajectory of the point or feature across frames will be satisfactorily smooth. The transmission module 240 can implement a trajectory analysis that includes generating a solution to a non-rigid non-linear transformation problem that transforms the pre-transition frame and/or the post-transition frame to match each other.

### 2.5 State Analysis Module

In one embodiment, the digital content processor 150 includes a state analysis module 250 configured to receive outputs from the state detection system 160. In other embodiments, the state analysis module 250 may be included in the state detection system 160. As described above, the state detection system 160 can monitor one or more states of a user using a plurality of sensors as the user experiences digital content provided via the HMD unit 112. The state analysis module 250 can process outputs from the state detection system 160 to assist in modification, particularly real-time modification, of the video stream. The state analysis module 250 can analyze one or more states of a user to determine whether the composition module 230 should modify (e.g., by a layer processing operation) the digital content currently being presented to a user. The state analysis module 250 may evaluate one or more conditions for modification to determine an appropriate modification of the content. In one embodiment, the state analysis module 250 compares one or more outputs associated with a state of a user to a threshold to determine the appropriate modification in relation to a treatment plan, described in greater detail below.

The state analysis module 250 can process captured audio associated with a user's interactions with the digital content and/or entities associated with the user (e.g., a therapist administering treatment to the user). As such, state analysis module 250 can generate a set of audio features that can be used to determine satisfaction of conditions for modification of digital content aspects. Rich information can be contained and processed from audible behaviors of the user in order to guide modification of the digital content. For instance, speech content, speech tone, or other speech characteristics of the user can indicate whether or not the user is ready to be presented with certain types of content through the VR platform 110.

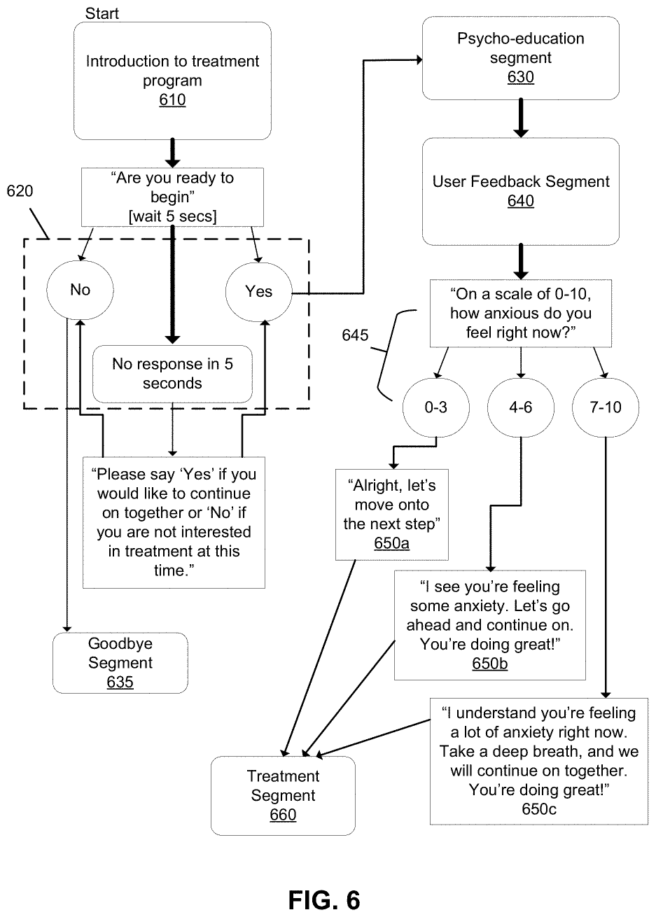

More specifically, the state analysis module 250 can process captured audio to extract features associated with speech, features of other vocal responses of the user or another entity, audio features from the environment, and/or any other suitable features. Speech features derived from the user or another entity can include speech content features extracted upon processing an audio signal to transform speech audio data to text data, and then applying a model (e.g., regular expression matching algorithm) to determine one or more components (e.g., subject matter, grammatical components, etc.) of captured speech content. Speech features can further include vocal tone features extracted upon processing an audio signal to transform audio data of speech or other user-generated sounds with a sentiment analysis. Speech features can also include quantitative features associated with length of a response, cadence of a response, word lengths of words used in a response, number of parties associated with a conversation, and/or any other suitable speech features. There can also be speech features including detected “expressions” or predefined phrases that a speech-to-text engine implemented by the digital content processor 150 can use to generate outputs. Examples of expressions are depicted in FIG. 6, in relation to number expressions associated with severity of patient state (e.g., of anxiety) or responses to yes/no questions. Environmental features can include audio features derived from sounds generated from objects in the environment of the user (e.g., in a clinical setting, in an indoor setting, in an outdoor setting, in an urban environment, in a rural environment, etc.) with audio comparison and/or classification algorithms.

The state analysis module 250 can additionally or alternatively process captured optical data associated with a user's interactions with the digital content and/or entities associated with the user (e.g., a therapist administering treatment to the user). As such, the state analysis module 250 can generate a set of optically-derived features that can be used to determine satisfaction of conditions for modification of digital content components. For example, the state analysis module 250 might receive information indicating that a user is smiling and hence this might satisfy a condition required to present a scenario to the user if the user appears to be happy. The visually-observed behaviors of the user can contain rich information useful for guiding modification of digital content. For instance, direction of the user's gaze or attention, facial expressions, or other stances or motions of the user can indicate whether or not the user is ready to be presented with certain types of content through the VR platform 110.

More specifically, the state analysis module 250 can process optical data to extract features associated with gaze of the user or other entity, facial expressions of the user or other entity, objects in the environment of the user, and/or any other suitable features. Gaze features can include features derived from processing optically-derived data of the user's eye(s) with eye tracking models (e.g., iris localization algorithms, pupil detection algorithms, etc.) to determine what the user is looking at in a VR environment or other environment associated with the provided digital content. The state analysis module 250 can use detection of objects (e.g., of interactive menus) that the user is looking at in a VR environment to provide modified content to a user. For instance, a menu or other visual interface can be provided to the user in a virtual environment associated with the digital content, and the state analysis module 250 can detect with the visual interface, through a state detection system 160 including an optical sensor, responses or interactions of the user. The state analysis module 250 can also determine facial expression features including features derived from processing optically-derived data of the user's face with expression determining models (e.g., classification algorithms to classify states of features of a user's face, feature fusion algorithms to combine individual input features into an output characterizing an expression, etc.). The state analysis module 250 can extract object-related features that include features of objects in the environment of the user upon processing optically-derived data with models having architecture for feature extraction and object classification in association with different types of objects.

The state analysis module 250 can additionally or alternatively process captured motion data (e.g., by inertial measurement units, accelerometers, gyroscopes, etc.) associated with a user's interactions with the digital content and/or entities associated with the user (e.g., a therapist administering treatment to the user). As such, the state analysis module 250 can generate a set of motion-associated features that can be used to determine satisfaction of conditions for modification of digital content components. For example, motions of the user's head or body can indicate whether or not the user is ready to be presented with certain types of content through the VR platform 110. Such sensors can be integrated within the HMD coupled to the user (e.g., in order to detect head or body motion), coupled to a controller (e.g., hand-coupled controller) associated with the VR platform, or interfaced with the user in any other suitable manner.

The state analysis module 250 can process the motion data to extract features associated with head motions of the user or other entity, body motions of the user or other entity, body configurations of the user or other entity, and/or any other suitable features. Head motion features can include features derived from processing motion sensor data derived from motion of the user's head with models (e.g., position models, velocity models, acceleration models, etc.) to determine what the user is looking at in a VR environment or other environment associated with the provided digital content. Head motion features can also be indicative of cognitive states in relation to gestures (e.g, head nodding, head shaking, etc.). Body motion features can include features derived from processing motion sensor data derived from motion of the user's body with models that characterize gestures (e.g., pointing gestures, shaking gestures, etc.) and/or actions (e.g., sitting, laying down, etc.) indicative of user states in reaction to provided digital content. The state analysis module 250 can detect objects (e.g., of interactive menus) that the user is pointing at or otherwise interacting with in a VR environment to provide modulated content to a user. For instance, a menu or other visual interface can be provided to the user in a virtual environment associated with the digital content, and detection of interaction with the visual interface, through a motion detection system included in the state detection system 160.

Furthermore, the state analysis module 250 can process captured biometric data associated with a user's interactions with the digital content and/or entities associated with the user (e.g., a therapist administering treatment to the user). As such, the state analysis module 250 can generate a set of biosignal-derived features that can be used to determine satisfaction of conditions for modification of digital content components. Biometric monitoring sensors implemented can include skin conductance/galvanic skin response (GSR) sensors, sensors for detecting cardiovascular parameters (e.g., radar-based sensors, photoplethysmography sensors, electrocardiogram sensors, sphygmomanometers, etc.), sensors for detecting respiratory parameters (e.g., plethysmography sensors, audio sensors, etc.), brain activity sensors (e.g., electroencephalography sensors, near-infrared spectroscopy sensors, etc.), body temperature sensors, and/or any other suitable biometric sensors. Such sensors can be integrated within the HMD coupled to the user (e.g., in order to detect head or body motion), coupled to a controller (e.g., hand-coupled controller) associated with the VR platform 110, or interfaced with the user in any other suitable manner.

The state analysis module 250 can use any of the above information to determine whether a user should receive content to increase or decrease his/her anxiety. Additionally, the state analysis module 250 can use any of the above information to select a video segment for presentation to a user, to track/evaluate user progress in treatment, to modify future video segments, or for any other relevant purposes. The state analysis module 250 can may provide information related to user progress to the clinician device 130 and/or the client device 120. In some embodiments, the state analysis module 250 compares the captured information to one or more thresholds to determine whether conditions for modification are satisfied. A method of modifying digital content is described in greater detail below in relation to FIG. 8.

3.0 Example Digital Content Capture System

FIG. 3 illustrates an example digital content capture system, in accordance with an embodiment. The example digital content capture system 140 shown in FIG. 3 includes a camera 310 having a 360-degree field of view in a horizontal plane (e.g., a plane parallel to the x-y plane). The camera 310 has a visual field that approximately covers a sphere, shown by the dashed lines. The omnidirectional camera system 300 includes a first mirror 320 opposing a lens 315 of the camera 310. The first mirror 320 has a first focal length and first radius of curvature. The first mirror 320 may be concave and centered within the field of view of the camera 310 (e.g., center of the mirror is aligned with the center of the lens 315 along the y-axis). The omnidirectional camera system 300 has a second mirror 330 with a second focal length and second radius of curvature. The second mirror 330 is positioned proximal to the camera 310 and has an aperture 332 centered about the lens of the camera. The second mirror 330 may also be concave. The second mirror 330 is positioned at least partially behind (e.g., along the y-axis) the lens 315 of the camera 310, in order to generate an omnidirectional video stream of a real-world environment. In a specific example, the omnidirectional camera system 300 include an 8K resolution 360 camera (e.g., PILOT ERA™, INSTA 360 PRO™, custom camera system, etc.), producing video content that has a 1-2 GB/minute storage requirement. As such, the video stream can be generated in an 8K resolution regime. In other embodiments, the omnidirectional camera system 300 can alternatively be any other suitable camera system.

### 4.0 Examples of Captured Content and Modified Content

FIG. 4A depicts an example of footage of an entity in front of a green screen, in accordance with an embodiment. The footage may be captured using the digital content capture system 140. The digital content capture system 140 can record an individual in front of a green screen 400 such that the video of the individual can be compiled with other digital content. The captured video of the individual may be dynamic content 410 (e.g., such that the dynamic content 410 is not replaced with still image pixels). The digital content capture system 140 can provide the video data to the digital content processor 150 to modify the footage and generate content for presentation to a user (e.g., as shown in FIG. 4C).

In one embodiment, the digital content processor 150 generates an alpha mask and color footage of the entity captured in FIG. 4A, shown in FIG. 4B. In the example shown in FIG. 4B, the alpha mask 420 and the colored footage 430 can be associated with a human entity. Pixels of the alpha mask associated with the entity have a value ranging from 1 (e.g., as white pixels) to 0 (e.g., as black pixels), where non-integer values of the mask indicate partial overlay (e.g., semi-transparency). The color footage may illustrate a more detailed image of the entity captured in FIG. 4A. The alpha mask 420 and/or the color footage 430 may be compiled with video content to generate a video stream. In some embodiments, the alpha mask 420 and/or the color footage 430 represents a layer of video content that may be combined with other layers for treatment of a mental health condition.

The footage captured in FIG. 4A and processed in FIG. 4B can be combined with additional video content to generate a video stream. FIG. 4C illustrates a frame of a modified video stream that may be provided to a user. In a specific example, a user with social anxiety may be provided with a room with a growing number of individuals. The user may first be presented with either a background panorama 440 or a stereo video 450 of FIG. 4C. In the example shown, both the background panorama 440 and the stereo video 450 include static and dynamic content. The dynamic content 410 includes the individual captured in FIG. 4A. The background of each scene includes one or more static region. As such, the background may be substantially the same across two or more frames, and the dynamic content processor 150 may apply stitching and blending operations to the frame of the video stream. In one embodiment, the background panorama 440 is preferred content to provide to a user, because the background panorama 440 illustrates a 360 degree environment that appears more realistic to a user. In other embodiments, the stereo video 450 may be preferable as it may be easier and/or faster to process by the VR platform 110. The background content and the dynamic content 410 may be associated with different layers. As such, the digital content processor 150 can apply one or more layer processing operations to adjust the anxiety that a user experiences. For example, the digital content processor 150 may apply an additional layer, adding additional individuals to the environment to increase anxiety of a user with social anxiety.

### 5.0 Example Digital Content Environment

As described above, in one embodiment, the digital content processor 150 can generate and/or modify content associated with a stress-inducing environment. FIGS. 5A-5E and illustrate example environments associated with operating a vehicle. The environment shown in FIGS. 5A-5E may be provided to a user who experiences anxiety when operating a vehicle. In one embodiment, the digital content processor 150 generates a video stream that transitions between the environments (e.g., from FIG. 5A to FIG. 5B to FIG. 5C). FIGS. 5A-5C and FIG. 5E each include static content and dynamic content. In the embodiment, the static content may be replaced with still image pixels by the composition module 230, as described above. The layers associated with FIGS. 5A-5E are generated in an 8K video regime, processed by the digital content processor 150 described above, and transmitted to the user dynamically through a 4K HMD of a VR platform 110.

FIG. 5A illustrates a frame of a video stream from the perspective of a driver, where the driver is traveling through a city. The digital content processor 150 may receive video content from a digital content capture system 140 that includes a sensor fixed to a vehicle and configured to capture content from the perspective of a driver. The digital content processor 150 can generate a video stream and identify one or more static regions of one or more frames of the video stream. In the embodiment of FIG. 5A, the digital content processor 150 identifies the dashboard as static content 510. The digital content processor 150 may apply a stitching and blending operation to each frame of the video stream to replace the static content 510 with still image pixels and blend the static content 510 and the dynamic content 520a to minimize perceived distortion. Thus, the user may experience a realistic and immersive environment of driving in a city using the VR platform 110.

The user may experience the video stream including frames of the environment comprising still image pixels and video pixels for a period of time, and the state detection system 160 may monitor one or more states of the user during the period of time. Additionally, a clinician using the clinician device 130 can monitor the user and determine the next environment to present to the user. The digital content processor 150 can receive information from the state detection system 160 and/or clinician device 130 during the period of time. The clinician may select an environment that increases or decreases anxiety levels of the user. In other embodiments, the VR platform 110 may automatically select an environment for the user based on one or more states of the user. For example, initially, the user may have an elevated heart rate while experiencing the VR environment shown in FIG. 5A. The VR platform 110 may select an environment after the user's heart rate subsides for a pre-defined period of time. For example, the VR platform 110 may select an environment that increases anxiety of the user after the user's heart rate has slowed to normal (e.g., 60 BPM) for a pre-defined period of time. In other embodiments, a clinician (or the VR platform 110) can prompt the user to determine if the user is ready to change environments, and use a controller of the VR platform 110 to trigger presentation of content of a new layer (e.g., of a bridge environment).

In one embodiment, the digital content processor 150 may transition the user between the environment shown in FIG. 5A to the environment shown in FIG. 5B to increase the user's anxiety. FIG. 5B illustrates an environment simulating a user driving over a bridge. The digital content processor 150 may modify the content to transition between the city view and the bridge view. In one embodiment, the layering module 236 applies one or more layer processing operations to transition between environments (e.g., the city view is a first layer and the bridge view is a second layer). The static content 510 may not change between frames. The static content 510 can thus include still image pixels substituted from a still image by the digital content processor 150, as described above. The digital content processor 150 can blend the new layer (e.g., dynamic content 520b illustrating the bridge environment) with the static content 510.

FIG. 5C illustrates a third environment associated with vehicle operation anxiety, in accordance with an embodiment. The digital content processor 150 can transition the video content from the environment shown in FIG. 5A or FIG. 5B to the environment shown in FIG. 5C. The user may experience increased or decreased anxiety driving through a tunnel compared to the city and the bridge. The static content 510 shown in FIG. 5C is also the dashboard. The dynamic content 520c illustrates the tunnel environment. The inclusion of static content 510 reduces the size of the video content, and allows the VR platform 110 to display a more realistic and immersive environment to the user.

In the example of FIGS. 5D-5E, the digital content processor 150 generates a visual effects layer to help a person overcome a driving-associated anxiety condition. In order to increase anxiety, the digital content processor 150 can add an additional layer to the environment shown in FIG. 5A. FIG. 5D illustrates a visual effects layer 530. The visual effects layer 530 includes rain and windshield wipers. The visual effects layer 530 characterizes how light is distorted by rain (e.g., on the windows of the vehicle). The digital content processor 150 generate a combined scene shown in FIG. 5E that includes the environment shown in FIG. 5A and the layer 530. FIG. 5E illustrates an environment that allows a user to experience driving through a city in rain. The digital content processor 150 can apply a stitching operation, a blending operation, a layer processing operation, or some combination thereof to generate the content shown in FIG. 5E. In other embodiments, the layer 530 may be combined with the environment shown in FIG. 5B or FIG. 5C. Additionally, in some embodiments, the user can experience just the layer 530 before experiencing the combined layers shown in FIG. 5E. Any of the above examples of layers used to generate modulated content, or variations thereof (e.g., variations of visual effects layers, variations in environments, etc.) can be combined in any other suitable manner for scene customization and presentation of content to users.

### 6.0 Example Segmentation of Content

FIG. 6 is a flow chart with a story that can branch at any point into two or more flows based on information associated with the user. For example, the story can branch based on voice (e.g., what the user is saying—speech to text plus regular expression matching; how the user says it—speech to text plus sentiment analysis, and length of response by the user), gaze (e.g., where the user is looking), controller (e.g., where the user is pointing), or tablet (e.g., administrator or therapist can move the user to a specific branch). The digital content processor 150 specifies which path the story takes based on these different interactions. For example, if a particular regular expression is spoken by a user and detected as a predefined phrase from a speech-to-text engine, the system will be trigger to transition to a particular video clip (e.g., psycho-education segment 630, treatment segment 660) in response. There are also predefined menus and visual indicators, such that the menus can provide alternative mechanisms for interaction and scene branching. The menus can be interacted with via gaze (e.g., focus on a menu item to select, optionally show “hover state” of the menu item when the user begins to focus over a menu item) or via speech. Each video segment from the defined flow chart can be cropped and saved to its own file. In one embodiment, the scene starts at a predefined starting video, and at the timestamp defined in the scene flow specification, a potential transition to a new scene is evaluated by various factors. For example, the state detection system 160 turns on the user's microphone, and listens for a particular phrase matching a regular express (“Please continue”) or is of a particular length of sentiment (a positive or happy statement), could determine if the user is pointing in a particular direction or within a bounded region, or could determine whether the user selected an item with a motion-based controller.

In the embodiment of FIG. 6, the digital content processor 150 can provide one or more video segments to a user based on the user response at different nodes. The segments describe different groupings of frames specific to one or more treatments. The segments (e.g., video clips) of content are connected by nodes, wherein interactions with a particular segment can be processed to appropriately modify or control what content is provided subsequent to a downstream node. FIG. 6 begins with a segment 610 including an introduction to a treatment program (e.g., for treating a phobia, PTSD, anxiety, etc.). The segment 610 can include a video clip of introductory content and can prompt an interaction with the user (e.g., “Are you ready to begin? Please say ‘Yes’ if you would like to continue”). The state detection system 160 can monitor the response of the user using one or more sensors (e.g., a microphone) and provide the information to the digital content processor 150. The digital content processor 150 determines the response of the user based on information from the state detection system 160 at node 620. If the user responds positively (e.g., by saying ‘Yes’), the digital content processor 150 can provide a subsequent segment based on the interaction (e.g., psycho-education segment 630). In other embodiments, if a response is not detected, the system may prompt additional interaction (e.g., “Please say ‘Yes’ if you would like to continue on together or ‘No’ if you are not interested in treatment at this time”). Alternatively, if the user responds negatively (e.g., by saying ‘No’), the digital content processor 150 can provide a goodbye segment 635.

After providing the psycho-education segment 630, the digital content may provide a user feedback segment 640. The user feedback segment 640 may prompt user interaction for the user to rank “On a scale of 0-10, how anxious do you feel right now?” Based on the user responses (e.g., 0-3, 4-6 or 7-10) at node 645, the digital content processor 150 can provide text content (e.g., 650a, 650b, 650c), such as encouragements or instructions to the user. The digital content processor 150 can subsequently provide a treatment segment 660 (e.g., examples shown in FIGS. 5A-5E). The digital content processor 150 may select a treatment segment based on the anxiety level indicated by the user. In other embodiments, the digital content processor 150 can provide the segments in any suitable order for treating a patient (e.g., psycho-education segment 630 can be provided after node 645). Adjacent video segments can be connected by nodes (e.g., 620, 645) associated with detection of different types of interactions (e.g., responses to questions prompted by the content, detected cognitive states, etc.) between the user and the content provided. As such, in some embodiments interactions can be at least partially guided by the segments of content, and content may be modified performed at pre-determined points defined by the nodes. The digital content processor 150 can also modify digital content at non-predefined nodes (e.g., nodes unassociated with prompting a specific type of interaction). However, the segments of the video stream can alternatively be structured in any other suitable manner.

As explained above, based on information collected at a node, the digital content processor 150 can provide a subsequent segment. To reduce latency when transitioning between video clips, the VR platform 110 can use multiple video decoders and frame buffers, where one can be currently playing, one can be loading the next video portion in the background, and then the VR platform 110 can switch between frame buffers only when the next video portion is pre-loaded. To improve fidelity of transitions and make transitions seamless, the digital content processor 150 and/or the VR platform 110 can determine which pair of frames between videos A and B can be connected most smoothly by defining pairwise distance or similarity metric between each frame of video frames A and B. The distance can be based on both the RGB color difference between the pair of video segments and the difference in optical flow (scene motion). The pair of video frames from A and B with minimal difference can be used to produce a transition with the least visual artifacts. Another way to improve fidelity of transitions is by tracking the x and y coordinates of points in the first and second video frames. The trajectory of these coordinates should be smooth across the transition. A non-linear distortion can be applied to the end of the first video segment and start of the second video segment to minimize any noticeable difference between the video frames. The system solves for the non-rigid, non-linear transformation that, when applied, will seamlessly morph a frame of video A to a frame of video B (e.g., allowing for seamless transition between segment 630 and segment 640).

### 7.0 First Example Method

FIG. 7 depicts a flowchart of a method 700 for generating digital content, in accordance with an embodiment. The method 700 functions to allow video content generated using a high-resolution system to be delivered through a system that only decodes lower resolution video content, such that delivery can be performed in a way that is still perceived by users as being high resolution content. In specific examples, outputs of the method can be used to generate digital content for treatment of mental health conditions. The digital content can be provided through virtual reality head mounted devices (HMDs) and/or other user output devices. The method 700 can also function to improve compression of video content delivered to users through output devices in a manner that allows for content delivery with mitigation of latency issues. The method 700 may be performed by one or more components of the system 100 described above. The method 700 can include fewer or greater steps than described herein. Additionally, the steps may be performed in a different order and/or by different entities.

The digital content processor 150 generates 710 a video stream of a real-world environment in a first video resolution regime (e.g., 8K resolution). The digital content processor 150 can receive real world video content from the content capture system 140 or some other storage system, and generate the video stream using the received content. In one embodiment, the digital content processor 150 generates the video stream before implementation of downstream portions of the method 700; however, the system can also generate/implement additional video content after implementation of at least some other portions of the method, such that the method 700 can include iterative generation and processing of video data. The digital content processor 150 can generate video streams of different lengths and/or different sizes. The digital content processor 150 generates a video stream that includes one or more frames, such as the frame illustrated in FIG. 5A.

The digital content processor 150 identifies 720 a set of static regions across frames of the video stream. The digital content processor 150 can identify static content in frames of a certain portion of the video stream or for the entire video stream. In the example of FIG. 5A shown above, as the user is driving through the city, the digital content processor 150 identifies the dashboard and steering wheel as static content. The digital content processor 150 can draw one or more boundaries around the static content and/or label the content as static. In other embodiments, the digital content processor 150 may alternatively or additionally identify and label one or more dynamic regions.

The digital content processor 150 can generate 730 a modified video stream by replacing the identified static regions with still image pixels using a stitching and blending operation. The digital content processor 150 can receive a still image from a camera system or a storage component. The still image may have a lower resolution (e.g., 4K resolution) than that of the video stream. In the example of FIG. 5A, the digital content processor 150 applies a stitching operation to replace the dashboard (i.e. the static content) with still image pixels. The digital content processor 150 applies a blending operation to blend the edges of the boundaries of the static content (e.g., near the edges of the dashboard) with the dynamic content (e.g., the city skyline) such that the frame appears cohesive and realistic to the user. The digital content processor 150 can apply the stitching and blending operations to one or more frames of the video content.

The digital content processor 150 transmits 740 the modified video stream to a display unit of a VR platform at a second resolution regime (e.g., 4K resolution). In one embodiment, the display unit is an HMD unit 112. Even with technical limitations of the VR platform 110, the VR platform 110 can decode the video stream with the replaced image pixels for presentation to a user such that the environment is realistic and immersive. In other embodiments, the digital content processor 150 can provide the video stream to another device capable of displaying the content.

In some embodiments, the digital content processor 150 can perform 735 one or more layer processing operations to transition the user to between environments. For example, as shown in FIGS. 5A and 5B, the digital content processor 150 may apply a layer processing operation to transition between the city skyline and the bridge environment. Part of the content can remain static (e.g., the dashboard) while the digital content processor 150 can substitute layers of dynamic content. The digital content processor 150 may perform operations related to increasing or decreasing anxiety of the patient when transitioning between different environments. The digital content processor 150 can modify the video stream responsive to input from the user (e.g., by the client device 120), from a clinician (e.g., by the clinician device 130), information from the state detection system 160, or information from the VR platform 110.

The operations described above can be performed subsequent to one another, or some operations may be performed in parallel. In some embodiments, the content may be modified based on dynamic feedback (e.g., from the clinician device 130, based on the state detection system 160), thus the steps may be performed out of order. In some embodiments, the steps may be performed in real time or near real time. The method 700 can include any other suitable steps for efficiently processing video content and/or other digital content generated in a first regime (e.g., 8K resolution, for transmission through devices subject to limitations of a second regime (e.g., 4K resolution). Additionally, the steps may be performed by different components of the system 100 or additional entities not shown in system 100.

### 8.0 Second Example Method

FIG. 8 depicts a flowchart of a method 800 for modifying digital content provided to a user based on one or more states of the user, in accordance with one or more embodiments. The digital content processor 150 can dynamically tailor features of content delivered to a user in an artificial reality environment (e.g., VR environment, AR environment, etc.), based upon one or more user states associated with detected behaviors. The digital content processor 150 can tailor content based upon observations of an entity (e.g., a therapist) associated with the user. In specific examples, digital content processor 150 can dynamically tailor content for treatment of mental health conditions, in order to improve outcomes associated with anxiety-related conditions, depression-related conditions, stress-related conditions, and/or any other conditions. Similarly, the content can also be tailored for any purpose in which a user might be placed in a different environment or situation, including for coaching or training of a user, for education, etc.

The method 800 depicted in FIG. 8 may include different or additional steps than those described, or the steps of the method may be performed in different orders than the order described. Furthermore, while VR devices are described in relation to applications of the method 800, the method 800 can be implemented with other output devices configured to provide digital content to users in any other suitable manner.

The digital content processor 150 transmits 810 digital content (e.g., video content) to a user via a VR platform 110. The digital content can include one or more segments (e.g., video clips), as described above in relation to FIG. 6. In some embodiments, the VR platform 110 provides introductory content to the user including modifiable components or features associated with affecting or measuring a mental health state of the user. Additionally, or alternatively, the digital content processor 150 transmits content to the user that can be associated with a stress-inducing environment. For example, for a user with driving related anxiety, the user may be presented with the environment shown in FIG. 5A.

The digital content processor 150 receives 820 an output from a state detection system 160 that monitors states of the user, as a user interacts with the digital content. As discussed above, the state detection system 160 automatically detects a user state, wherein one or more detected user states associated with user interaction with provided content can be used to modify aspects of content provided to the user dynamically and in near-real time. As such, the digital content processor 150 can dynamically modify provided content in a time critical manner (e.g., in relation to optimizing outcomes of a therapy session for a mental health condition). The dynamic modification can also include selecting different segments of content from a library of options, each segment having different features or modifications that may be appropriate for the current state of the user that was detected. In some embodiments, the digital content processor 150 can also receive information from an entity associated with the user (e.g., a coach entity, a therapist entity) via the clinician device 130 in communication with the VR platform 110 as the user interacts with digital content. For example, a therapist can sit in the room with the user and observe the user's reactions, and can also control the content provided accordingly via communication between the clinician device 130, the digital content processor 150, and the VR platform 110. The therapist may be able to view the digital content provided to the user via the VR platform 110 on the clinician device 130. In some embodiments, the therapist can select different scenarios to provide to the user such as busy city traffic and highway tunnels in the example of FIG. 5A-5C. The therapist can also select the weather conditions, such as clear or rainy, as shown in FIGS. 5D-5E. The digital content processor 150 can modify the video stream based on input from the clinician device 130. Thus, the therapist or coach can adjust what content is presented to the user during the user's virtual experience based on how the user is responding to the content or based on certain actions that a user takes while experiencing the content.

The digital content processor 150 generates 830 a modified component of the digital based on information received from the state detection system 160 and/or the clinician device 130. In other words, the system generates the modified digital content based on feedback or user interactions that have occurred with the content that was previously shown. As such, the digital content processor 150 analyzes one or more states of the user to determine what feature(s) of the digital content should be adjusted by executing content control instructions in a computer-readable format. As such, if information received from the state detection system 160 and/or the clinician device 130 indicate that the user is in a state that would be appropriate for interacting with more stressful content, the digital content processor 150 can generate this modified content for presentation user. The generation 830 of the modified component or segment can also include selecting from different options for pre-generated content. Conversely, if information received from the state detection system 160 and/or the clinician device 130 indicate that the user is in a state that would be appropriate for interacting with less stressful content, similarly the system can generate and provide content that is less stressful to the user. Finally, if information received from the state detection system 160 and/or the clinician device 130 indicate that the user should have content similar to what the user is currently viewing in terms of level of stress caused by the content, this again can be provided to the user. In some embodiments, information received from the state detection system 160 and/or the clinician device may be compared to one or more thresholds or conditions for modification, to determine whether the digital content processor 150 should modify the digital content.

Additionally, the digital content processor 150 can perform 835 a layer processing operation (e.g., involving decomposition and processing of different regions of video/imagery) to incorporate a layer based on user states captured in the outputs, which can be an example of a modification made to a segment of content. As described above, the layer processing operation can include performing stitching and blending operations associated with each layer of modulated content. In relation to mental health promoting therapies and presentation of stress-associated environments with various levels of severity in stressful situations, the digital content processor 150 can incorporate layers corresponding to an increase in stress severity or decrease in stress severity. Thus, the user can be presented with more or less stressful content based on the user's current state or an anticipated future state in relation to improving the user's ability to cope with anxiety (e.g., for an anxiety related disorder). For example, in the embodiment of FIGS. 5D-5E, the digital content processor 150 can apply a layer processing operation to incorporate the layer shown in FIG. 5D into FIG. 5A. The layer processing operation can generate a frame shown in FIG. 5E.

In one example, the digital content processor 150 can process information from the state detection system 160 to determine a “path” that the subsequent segments of digital content (e.g., segments of digital content not yet experienced by the user) can take, as discussed above in relation to FIG. 6. The digital content processor 150 can modify content of the subsequent segments with a layer processing operation, where layers are ultimately composited and presented to the user at the HMD unit 112 or other display. In the specific example, where segments of content are connected by nodes associated with transition points in content, the digital content processor 150 can activate one or more subsystems of the state detection system 160 as a node is approached, receive digital outputs from the subsystem(s), and process the outputs against conditions to determine if or how content of segments downstream from the node should be modified (e.g., in relation to stressor severity) based on user state or behavior.

In a specific example, the digital content processor 150 activates a user's microphone and the digital content processor 150 processes audio content. For example, the system can identify a phrase that matches a regular expression, of a phrase that satisfies a length condition, or of a sentiment or tone carried in the audio data. These identified phrases or sentiments can guide modification of subsequently provided content with the layer processing operation to generate layers that are composited during playback at the HMD unit 112. In another specific example, an optical detection subsystem and/or a motion sensor can be activated and the system can extract and process gaze-related information (e.g., from eye tracking, from head movement information, etc.) for identification of a direction in which the user is looking in order to control what content is presented next. As a further example, a motion capture subsystem or other input device can be activated and the system can extract/process activity data (e.g., object selection information, pointing information, etc.) for identification of activity characteristics (e.g., the user is standing, walking, running, dancing, etc.) in order to guide modification of the next content to be provided with the layer processing operation.

The digital content processor 150 can modify digital content and timely transmit modified content to the user, based on user state, without latency issues or reduction in fidelity of transitions between segments of content. The digital content processor 150 transmits 840 at least one segment of the digital content, with the modified component, to the user at the HMD unit 112. The digital content processor 150 functions to provide modified segments of digital content to the user in a timely manner, in relation to improving outcomes (e.g., treatment outcomes) and/or user experiences with provided digital content based on user state. The state detection system 160 can continue to monitor the user as the user interacts with the modified content. In some embodiments, the digital content processor 150 receives 820 additional outputs from the state detection system 160 as the user interacts with the modified content, such that the content can be continuously updated in near real time.

The digital content processor 150 can additionally include functionality for reducing latency associated with transitioning between different segments of digital content and/or latency associated with compositing of multiple layers of components when presenting modified content to the user at the HMD unit 112 or other display. Thus, the digital content processor 150 can composite 845 video/image data modified by the digital content processor 150 during presentation or playback of content to the user at the HMD unit 112 or other display. As such, the digital content processor 150 can be used to dynamically provide video clips with layers appropriate to the user's cognitive state, without buffering or other issues. Additionally or alternatively, the digital content processor 150 can store content on the HMD unit 112 or other device associated with a display such that the content can be accessed in real time (or near real time) in relation to a user session where content is provided to the user.

### 9. Conclusion

The system 100 and methods 700 and 800 can confer benefits and/or technological improvements, several of which are described below. The system 100 and methods 700 and 800 can produce rich digital content in a high resolution video data regime that can be that can be delivered through systems not typically capable of decoding or delivering such content without reductions in performance (e.g., latency-related performance, resolution-related performance, etc.). As such, the system 100 and methods 700 and 800 can improve function of systems used in VR platforms relation to improved content delivery through devices that are subject to resolution limitations, latency-related issues, and compression issues when normally handling content from the high-resolution regime.

The system 100 and methods 700 and 800 can additionally provide novel tools in an industry fraught with inefficiencies in relation to waitlists to receive treatment and limited treatment options, thereby improving treatment outcomes with dynamic tools not capable of being delivered entirely by human entities. The system 100 and methods 700 and 800 can promote user engagement and reduce therapist burden, increasing effectiveness of treatment of mental health conditions using digital therapeutics. Furthermore, the system 100 and methods 700 and 800 allow for dynamic presentation of content in near real time, allowing content to be tailored to a patient.

The system 100 and methods 700 and 800 can additionally efficiently process large quantities of data (e.g., video data) by using a streamlined processing pipeline. Such operations can improve computational performance for data in a way that has not been previously achieved, and could never be performed efficiently by a human. Such operations can additionally improve function of a system for delivering content to a user, wherein enhancements to performance of the online system provide improved functionality and application features to users of the online system. As such, the system 100 and methods 700 and 800 can provide several technological improvements.

The system 100 and methods 700 and 800 described above are related to the context of treatment of mental health conditions, however, in other embodiments the embodiments described herein may be useful for other applications. Additionally, the system 100 may include fewer or greater components than described herein, or the components of the system 100 may have be configured to perform alternative functions.

The foregoing description of the embodiments has been presented for the purpose of illustration; it is not intended to be exhaustive or to limit the patent rights to the precise forms disclosed. Persons skilled in the relevant art can appreciate that many modifications and variations are possible in light of the above disclosure.

Some portions of this description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described.

Embodiments may also relate to an apparatus for performing the operations herein. This apparatus may be specially constructed for the required purposes. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments may also relate to a product that is produced by a computing process described herein. Such a product may comprise information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights, which is set forth in the following claims.

What is claimed is:

1. A method of modifying a video stream for presentation to a user, the method comprising:
generating a video stream in a first video resolution regime for display on a head mounted device associated with a virtual reality platform;
identifying a static region and a dynamic region in a frame of the video stream, the static region and the dynamic region each comprising a plurality of video pixels;
substituting one or more of the plurality of video pixels associated with the static region with one or more still image pixels;
applying a blending operation at a boundary of the static region to generate a modified frame, the blending operation configured to blend the one or more still image pixels and one or more of the plurality of video pixels associated with the dynamic region;
generating a modified video stream including the modified frame; and
transmitting the modified video stream in a second video resolution regime to the virtual reality platform for display on the head mounted device, wherein the second video resolution regime is smaller than the first video resolution regime.

2. The method of claim 1, further comprising:
receiving video data for generating the video stream from an omni-directional camera with a 360-degree field of view in a horizontal plane.

3. The method of claim 1, wherein the video stream comprises content for treating a mental health condition.

4. The method of claim 1 wherein identifying the static region comprises:
implementing one or more computer vision techniques from a group consisting of: optical flow techniques, background subtraction techniques, frame-difference techniques, and temporal difference techniques.

5. The method of claim 1, further comprising:
processing the static region into a two-dimensional texture; and
processing the dynamic region into one or more two-dimensional textures.

6. The method of claim 5, wherein substituting one or more of the plurality of video pixels associated with the static region with one or more still image pixels comprises:
converting a ray corresponding to a still image pixel to coordinates of an equirectangular texture, wherein the coordinates of the equirectangular texture correspond to a two-dimensional texture coordinate;
converting the coordinates of the equirectangular texture to video frame coordinates in a video frame coordinate space; and
responsive to determining that the video frame coordinates of the ray fall within a pre-defined range of the video frame coordinate space, indexing the still image pixel corresponding to the ray in the frame at the computed coordinates in the frame coordinate space.

7. The method of claim 1, wherein applying the blending operation comprises:
applying a color correction operation to the one or more still image pixels.

8. The method of claim 1, further comprising:
applying a layer processing operation to the video stream, the layer processing operation configured to incorporate a layer in the video stream associated with treating a mental health condition.

9. The method of claim 8, wherein the incorporated layer is configured to increase anxiety of the user.

10. The method of claim 1, wherein the first video resolution regime comprises 8K resolution and the second video resolution regime comprises 4K resolution.

11. The method of claim 1, wherein substituting one or more of the plurality of video pixels associated with the static region with one or more still image pixels comprises:
applying a homography matrix operation configured to transform features of the still image to fit the static region of the frame.

12. A system for providing therapeutic digital content to a user, the system comprising:
a virtual reality platform comprising a head mounted display unit configured to display digital content; and
a processor, and a computer readable storage medium storing code that when executed by the processor, causes the processor to:
receive a video stream in a first video resolution regime for display on the head mounted device;
identify a static region in a frame of the video stream, the static region comprising a plurality of video pixels;
substitute one or more of the plurality of video pixels associated with the static region with one or more still image pixels to generate a modified frame;
generate a modified video stream including the modified frame; and
transmit the modified video stream in a second video resolution regime to the virtual reality platform for display on the head mounted device, wherein the second video resolution regime is smaller than the first video resolution regime.

13. The system of claim 12, further comprising:
a state detection system comprising one or more sensors configured monitor one or more states of the user.

14. The system of claim 13, wherein the computer readable medium further stores code that when executed by the processor causes the processor to:
apply a layer processing operation to the video stream based on one or more states of the user, the layer processing operation configured to incorporate a layer in the video stream.

15. The system of claim 14, wherein the incorporated layer is configured to increase anxiety of the user.

16. The system of claim 12, wherein substituting one or more of the plurality of video pixels comprises:
applying at least one of: an image alignment process, a feature alignment process, a registration process, or a homography matrix operation.

17. The system of claim 12, further comprising:
an omni-directional camera system configured to:
capture a video stream of a real-world environment; and
provide the video stream to the processor.

18. The system of claim 12, wherein the video stream comprises content for treating a mental health condition.

19. The system of claim 12, wherein generating a modified video stream including the modified frame comprises:
applying a blending operation at a boundary of the static region, the blending operation configured to blend the one or more still image pixels and one or video pixels not included in the static region.

20. The system of claim 12, wherein the first video resolution regime comprises 8K resolution and the second video resolution regime comprises 4K resolution

21. A computer-readable storage medium having instructions encoded thereon that, when executed by a processor, cause the processor to:
generate a video stream in a first video resolution regime for display on a head mounted device associated with a virtual reality platform;
identify a static region and a dynamic region in a frame of the video stream, the static region and the dynamic region each comprising a plurality of video pixels;
substitute one or more of the plurality of video pixels associated with the static region with one or more still image pixels;
apply a blending operation at a boundary of the static region to generate a modified frame, the blending operation configured to blend the one or more still image pixels and one or more of the plurality of video pixels associated with the dynamic region;
generate a modified video stream including the modified frame; and
transmit the modified video stream in a second video resolution regime to the virtual reality platform for display on the head mounted device, wherein the second video resolution regime is smaller than the first video resolution regime.
